(19) United States
(12) Patent Application Publication
(10) Pub. No.: US 2017/0156943 A1
(43) Pub. Date: Jun. 8, 2017

(54) Incontinence Guard

(71) Applicant: Sangeeta Muthu, Sherman Oaks, CA (US)

(72) Inventor: Sangeeta Muthu, Sherman Oaks, CA (US)

(21) Appl. No.: 14/960,130

(22) Filed: Dec. 4, 2015

(51) Int. Cl. A61F 13/505; A61F 13/476; A61F 13/49

(52) U.S. Cl. CPC A61F 13/505; A61F 13/49061; A61F 13/49004; A61F 13/476; A61F 2013/15121

(57) ABSTRACT

An incontinence guard, or diaper, is provided which comprises a girdle portion and a waste containment portion. The girdle comprises at least one flap having means to releasably secure itself to an outward-facing side of the waste containment portion and is moreover elastic so as to forcibly secure the waste containment portion around a wearer's waist. In use, a soiled waste containment portion may simply be detached from the girdle and replaced with a clean waste containment portion while the girdle remains secured around the wearer's waist. Alternatively, only a portion of the waste containment portion may be detatched from the girdle to permit changing of the soiled absorbent layer alone. This avoids a need to place the wearer in a lying position, or otherwise require the wearer to meaningfully cooperate, such as by responsively articulating from his hips and knees, when a caregiver undertakes to replace soiled incontinence guards.

### GOVERNMENT CONTRACT

Not applicable.

### CROSS-REFERENCE TO RELATED APPLICATIONS

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material that is subject to copyright protection. This patent document may show and/or describe matter that is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to disposable incontinence garments and, more particularly, to diapers having distinct and fully separable portions which permit a wearer or caregiver to change a soiled diaper even when a child refuses to lay down or otherwise cooperate with such changing.

### BACKGROUND

Various different garments are well known for guarding against incontinence in human babies, senior citizens, those having conditions affecting bowel movements, and even animals. In many cases, the incontinence guard or diaper is formed as a single piece that fits between a wearer's legs and is securable around the waist of a wearer. For example, U.S. Pat. No. 3,089,494 to Schwartz teaches a pinless, absorbent diaper with oppositely extending wings pressable into sealed engagement with an upwardly folded portion to hold the diaper in place. As another example, U.S. Pat. No. 4,402,688 to Julemont teaches a similarly adhesively securable, disposable diaper having contoured elastic portions which allow the diaper to be form-fitting. As yet another example, U.S. Pat. No. 8,518,007 to Labit teaches a diaper that is similarly shaped to the aforementioned art but comprises material suited to reuse.

In use, these types of diapers are opened along flat surface, and the wearer is laid in a supine position so that a rear portion of the diaper is positioned underneath the wearer's pelvis. Then, a front portion of the diaper is moved between the wearer's legs to rest on the wearer's abdomen and the entire assembly is secured in place with means that connect the rear portion and front portion around the wearer's waist. It is commonly known, however, that some wearers, and in particular human babies and toddlers, can be uncooperative during this diaper-changing process. Indeed, they often kick their legs, attempt to roll away, and even manage to run off before a caregiver has optimally secured the fresh diaper to the baby.

There have been some attempts to simplify the diaper-changing process. For instance, U.S. Pat. No. 5,858,012 to Yamaki et al. and U.S. Pat. No. 6,423,042 to Sasaki disclose pants-type disposable diapers formed to be stepped into and pulled up around the waist of a wearer. Still, these solutions are deficient because they require a wearer to cooperatively articulate from his knees and hips during the changing process. Young wearers, such as babies and toddlers, are often unwilling to do so, and other's may be physically unable. With both traditional and simplified diapers, a baby wearer may be sleeping when his caregiver realizes that a soiled diaper must be changed. Changing the diaper while allowing the baby to remain asleep can be a challenge however, because, using either the common wrap diapers or even pull-up variety, the parent or caregiver must move the baby's legs and buttocks to remove and replace it. Such jostling often disturbs the baby, causing him to wake up during the process.

As such, although various proposals have been made to solve the problem, none of those in existence combine the characteristics of the present invention. Therefore, there is a need for a means for an improved incontinence guard or diaper.

### SUMMARY

The present disclosure is directed to an incontinence garment or guard, such as a diaper, which is configured to avoid requiring the wearer to take a supine position while replacing garments soiled with bodily waste. Indeed, certain features of the garment further permit replacement of soiled portions of the garment while avoiding the need for the wearer to articulate his or her limbs or otherwise exhibit a range of motion at all, allowing a caregiver to replace soiled garments whether or not the wearer cares or is able to cooperate with the changing.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In an embodiment, the incontinence guard, garment, or even baby diaper, comprises a waste containment portion and a girdle. More particularly, the girdle—or waist band—may be sized to encircle a wearer's waist. At least a front portion of the waste containment portion is releasably securable to the girdle portion so that when soiled, it may be replaced with a fresh one independent of a need to entirely remove the incontinence guard, or even the girdle, from the wearer's body. Thus, for instance, in some embodiments, the front portion of the waste containment portion may be released from securement to the girdle, an absorbent and removable pad may be taken from within the waste containment portion, and an unsoiled absorbent pad may be placed in its stead, all while avoiding a need to remove any portion of the incontinence guard from the wearer.

In other embodiments, the waste containment portion and girdle are wholly separate pieces that are releasably securable to one another. For example, in some embodiments, the entire waste containment portion may be released from securement to the girdle, and an unsoiled, absorbent, and releasably securable waste containment portion may be placed in its stead, all while avoiding a need to remove the entire assembly defined by the girdle and waste containment portion from the wearer. That is, the girdle remains in place around a wearer's waist when the waste containment portion is fully removed for changing.

It is contemplated that arranging the elements of the incontinence guard according to the various embodiments permits a caregiver or the wearer himself to replace soiled garments with little effort. Indeed, in the event that a wearer is a baby or toddler, or even pet, the caregiver may replace a soiled garment regardless of the position that the wearer has taken. Indeed, the wearer may be standing or laying down and need not be facing the caregiver at all during the changing procedure.

In an embodiment, the waste containment portion may comprise layered materials and have a front end and a rear end spaced apart by opposing and elastic first and second sides. The layers may comprise a wearer-facing liquid-pervious layer, an outward-facing liquid-impervious layer, and an absorbent layer between the liquid pervious and impervious layers. Configured as such, it is contemplated that any liquid waste passed by the wearer may pass through the liquid-pervious layer in contact with his body to be absorbed by the absorbent layer beneath. The outward-facing liquid-impervious layer, then, ensures that such waste remains within the absorbent layer and does not leak out of the incontinence guard.

The girdle, being sized to encircle the wearer's waist, may have a wearer-facing side, an outward-facing side, a top edge, a bottom edge, and one or more flaps extending downward from the bottom edge. The girdle may further comprise means for releasably securing at least the front edge of the outward-facing liquid-impervious layer of the waste containment portion to the wearer-facing side of the one or more flaps. For example, in such embodiments, a rear end of the waste containment portion may be permanently secured to the bottom edge of the girdle so that, to replace soiled garments, the front end of the waste containment portion is unsecured from the bottom flap of the girdle to expose the soiled portion of the garment. In such an embodiment, the absorbent layer may be removable from between the liquid-pervious and impervious layers, while the girdle remains secured around the wearer's waist, from a slot formed in the liquid-pervious layer and sized to receive the removable absorbent layer. Alternatively the absorbent layer may be removeably adhesible to or meshable with any wearer-facing side of the waste containment portion so that, when soiled, the absorbent layer may simply be peeled off of the waste containment portion. Once removed, the soiled absorbent layer may then replaced, in like fashion, with an unsoiled absorbent layer.

In some embodiments, the girdle may even further comprise means for releasably securing the rear end of the outward-facing liquid-impervious layer of the waste containment portion to the wearer-facing side of the one or more flaps so that the girdle and waste containment portion comprise wholly separable pieces. One skilled in the art, however, will recognize that instead, the rear end only, rather than the front end, may be releasably securable to the one or more flaps of the girdle without departing from the invention. That is, the front end of the waste containment portion may instead be permanently secured to the bottom edge of the girdle so that, to replace soiled garments, the rear end of the waste containment portion is unsecured from the bottom flap of the girdle to expose the soiled portion of the garment. The foregoing is thus offered by way of example only and not of limitation.

In some embodiments, a single flap may be fully integrated into the body of the girdle so that the girdle and flap form a continuous unit. In another embodiment, the one or more flaps may elastically extend downward from the bottom edge of the girdle. Means for releasably securing the one or more flaps and waste containment portion may comprise at least one of hook and loop fasteners, adhesive tape, hook and eye closures, and snaps.

The girdle may comprise elastic material to forcibly maintain the front and rear ends of the waste containment portion against the body of the wearer. Indeed, the girdle may be formed as an elastic and continuous band which may be pulled up around the wearer's waist. In another embodiment, though, the girdle comprises a first end longitudinally spaced from a second end, and at least one means for adjustably securing the first end and second end to one another around the wearer's waist.

The at least one means for adjustably securing first and second ends of the girdle may comprise tabs that are any of meshable, mateable, or adhesible to the outer-facing side of the girdle. The tabs may elastically extend from the first and second ends of the girdle. It is also contemplated, however, that the the tabs may be integrated into the body of the girdle so that the means for adjustably securing the first end and second end to one another is on the wearer-facing side of either one of the ends, permitting releasable securement of such end to the outer-facing side of the other. It should be noted that the means for adjustably securing the first end and second end of the girdle can align with any possible portion of the wearer's body when the girdle is worn. As an example, when secured around the wearer's waist, such means may secure the girdle in line with the wearer's hips, his navel, above his buttocks, or any other portion of the wearer's body.

In some embodiments, the girdle may be so elastic as to permit the length of the girdle to be stretched to accommodate wearers of various girths. In other embodiments, girdles may sized to accommodate wearers of particular girth.

The width of the girdle may vary as well. For instance, one skilled in the art will recognize that some widths may fit more comfortably against the body of the wearer. Thus, it is contemplated that an elastic girdle about 2 inches to about 5 inches wide may disperse elastic force over a greater surface area on the wearer, making the girdle more comfortable for extended wear. Of course, one skilled in the art will recognize that various dimensions discussed herein are provided by way of example only and not of limitation. Indeed, features such as length, width, and thickness may be chosen according to particular needs of the wearer and may even depend on the particular materials chosen to comprise either or both of the girdle and waste containment portions.

Thus, it is an object of the invention to provide a disposable or reusable incontinence guard configured to permit replacement of soiled portions regardless of the wearer's physical position.

One or more of the above-disclosed embodiments, in addition to certain alternatives, are provided in further detail below with reference to the attached figures. The disclosed subject matter is not, however, limited to any particular embodiment disclosed.

### BRIEF DESCRIPTION OF THE DRAWINGS

FIG. 1 shows a plan view of a girdle in accordance with one embodiment of the invention.

FIG. 2 shows a perspective view of the girdle from FIG. 1.

FIG. 3 shows a perspective view of an assembled girdle in accordance with one embodiment of the invention.

FIG. 4A shows a perspective view of a girdle in accordance with another embodiment of the invention.

FIG. 4B shows a perspective view of a girdle in accordance with yet another embodiment of the invention.

FIG. 5 shows a plan view of a waste containment portion in accordance with one embodiment of the invention.

FIG. 6 shows an alternate plan view of the waste containment portion from FIG. 5.

FIG. 7 shows a perspective view of the waste containment portion in accordance with one embodiment of the invention.

FIG. 8 shows a girdle and waste containment portion assembled to define an incontinence guard in accordance with one embodiment of the invention.

FIG. 9 shows a girdle and waste containment portion assembled to define an incontinence guard in accordance with another embodiment of the invention.

The disclosed embodiments may be better understood by referring to the figures in the attached drawings, as provided below. The attached figures are provided as non-limiting examples for providing an enabling description of the apparatus claimed. Attention is called to the fact, however, that the appended drawings illustrate only typical embodiments of this invention and are therefore not to be considered as limiting of its scope. One skilled in the art will understand that the invention may be practiced without some of the details included in order to provide a thorough enabling description of such embodiments. Well-known structures and functions have not been shown or described in detail to avoid unnecessarily obscuring the description of the embodiments.

### DETAILED DESCRIPTION

Having summarized various aspects of the present disclosure, reference will now be made in detail to that which is illustrated in the drawings. While the disclosure will be described in connection with these drawings, there is no intent to limit it to the embodiment or embodiments disclosed herein. Rather, the intent is to cover all alternatives, modifications and equivalents included within the spirit and scope of the disclosure as defined by the appended claims.

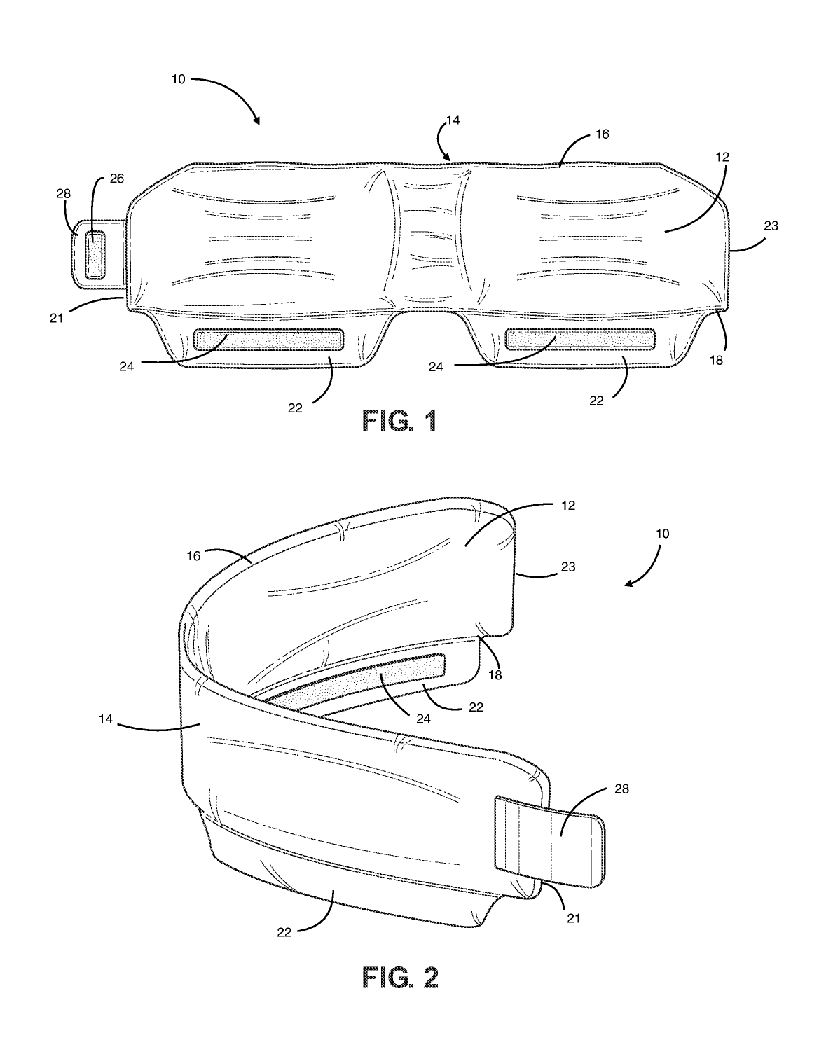

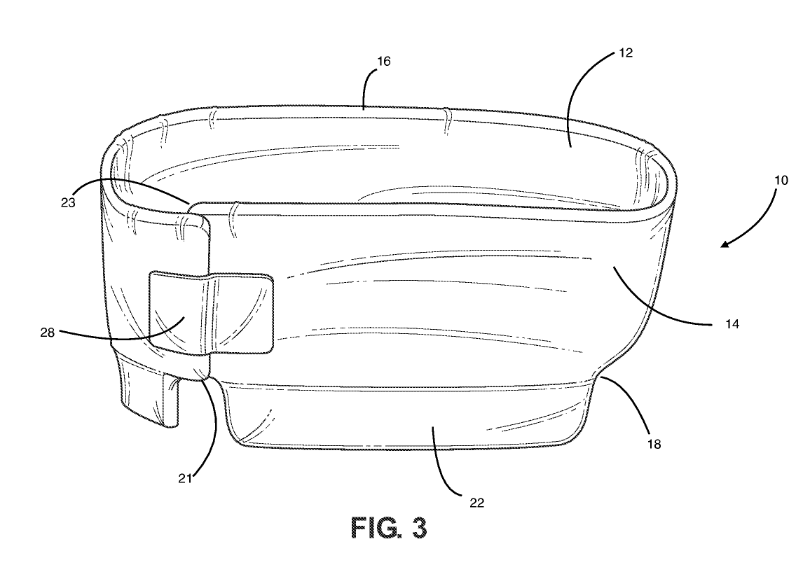

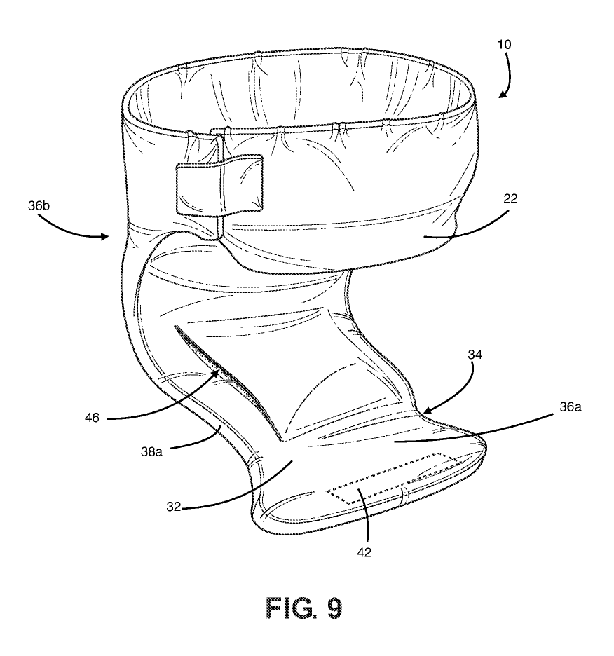

The improved incontinence guard comprises a girdle and a waste containment portion. FIGS. 1-4 show a girdle 10 that is separate from a waste containment portion, whereas FIG. 9 illustrates a waste containment portion 30 that is integrated with a girdle 10. For the sake of clarity, the waste containment portion and girdle will be described, in part, as separate units below, however, it will be recognized that both embodiments are contemplated and enabled by the disclosure.

With reference now to FIGS. 1 and 2, the girdle 10 may comprise a wearer-facing side 12, an outward-facing side 14, a top edge 16, a bottom edge 18, and one or more flaps extending downward from the bottom edge 22. The girdle 10 may further comprise means for releasably securing 24 the waste containment portion (discussed in further detail below) to the wearer-facing side 12 of the one or more flaps 22.

The girdle 10—or waist band—may be sized to encircle a wearer's waist. It may comprise elastic material configured to forcibly maintain the waste containment portion against the body of the wearer. In some embodiments, the girdle 10 may even be so elastic as to permit the length of the girdle 10 to be stretched to accommodate wearers of various girths. In other embodiments, the girdle 10 may be relatively inelastic and sized to accommodate wearers of particular girth. For example, and without limitation, the girdle 10 may comprise elastic webbing, or disposable stretchy plastics such as thermoplastic polyurethane. It is also contemplated that the girdle 10 may comprise rugged materials capable of withstanding multiple washings and reuse such as lycra, spandex and other natural and man-made materials known in the art to be suitable for use as a waist-band or girdle.

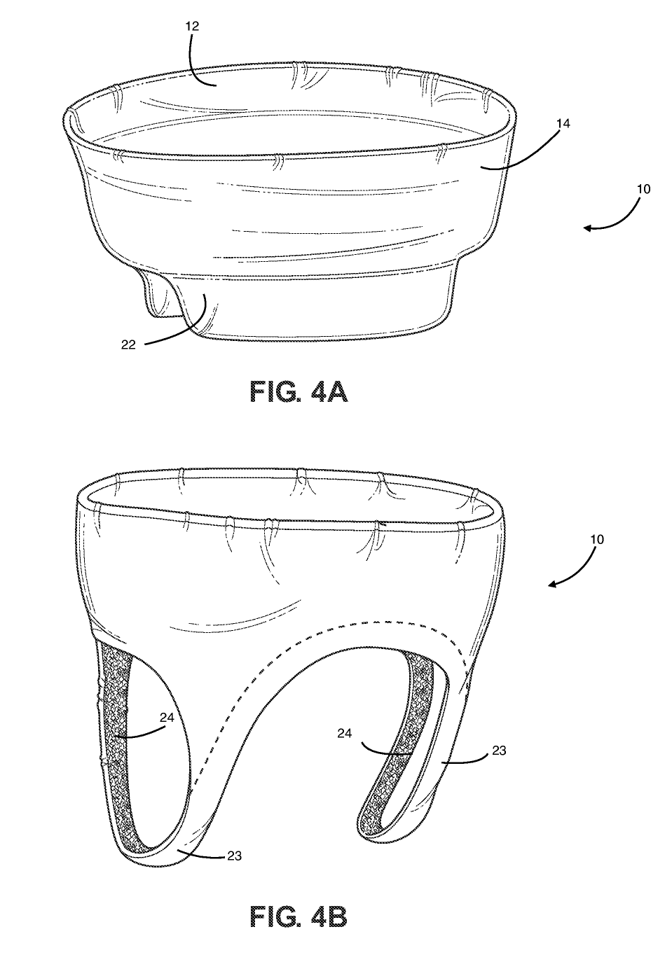

As shown in FIG. 4A, in one embodiment, the girdle 10 may be formed as an elastic and continuous band which may be pulled up around the wearer's waist. Such elasticity may ensure that the girdle 10 formed as a stretchy band is capable of being resiliently pulled over the wearer's hips—which are typically wider than his waist—and then forcibly maintaining a snug position around the wearer's waist or lower abdomen.

As illustrated in FIGS. 1, 2, and 4A-B, however, in another embodiment, the girdle 10 may comprise a first end 21 longitudinally spaced from a second end 23, and at least one means for adjustably securing 26 the first end 21 and second end 23 to one another around the wearer's waist. In such an embodiment, the girdle 10 may comprise elastic or inelastic material owing to the fact that such means for adjustably securing 26 the first end 21 and second end 23 around the wearer's waist may be disengaged to loosen or tighten the girdle 10 in its position around the wearer.

The at least one means for adjustably securing 26 first and second ends 21, 23 of the girdle 10 may be formed on or integrated with one or more tabs 28 that are any of meshable, mateable, or adhesible to the outer-facing side of the girdle. The tabs 28 may elastically extend from the first and second ends of the girdle further supporting the adjustability of the girdle 10 around the wearer's waist. It is also contemplated, however, that the one or more tabs 28 may be integrated into the body of the girdle so that the means for adjustably securing 26 the first end and second end 21, 23 to one another is on the wearer-facing side 12 of either one of the ends 21, 23, permitting releasable securement of such end to the outer-facing side 14 of the other.

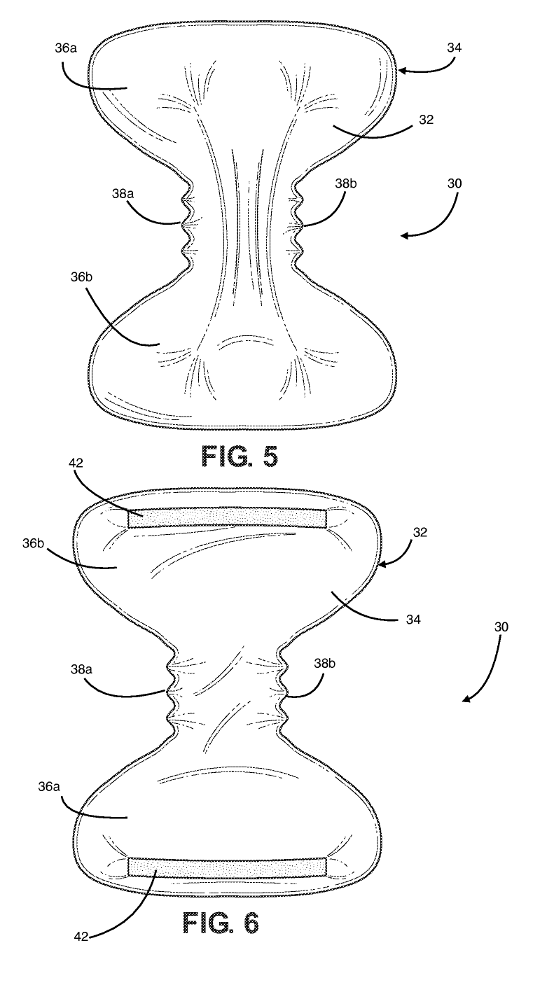

Turning attention to embodiments of the waste containment portion 30 illustrated in FIGS. 5 through 7 the waste containment portion 30 may have layered materials comprising a wearer-facing liquid-pervious layer 32, an outward-facing liquid-impervious layer 34, and an absorbent layer between the liquid pervious and impervious layers. Configured as such, it is contemplated that any liquid bodily waste passed by the wearer may pass through the liquid-pervious layer 32 in contact with his body to be absorbed by the absorbent layer beneath. Thus, in some embodiments, the wearer-facing liquid-pervious layer 32 may comprise any or a combination of polyester, fleece, naturally bacteria resistant materials such as wool, and any other porous material configured to permit liquid to pass through the garment to the absorbent layer, allowing the wearer's skin to stay comfortable and dry. It is additionally contemplated that this may prevent chaffing, infection, and other discomfort over periods of extended wear. Indeed, the wearer-facing liquid-pervious layer 32 may further comprise enhancements such as aloe, vitamin E, other compounds known to soothe and protect skin in direct contact with the incontinence guard, decorative dyes, and even fragrance to mask the smell of bodily waste.

The outward-facing liquid-impervious layer 34, then, ensures that such waste remains within the absorbent layer and does not leak out of the incontinence guard. For example, and without limitation, to ensure that waste is effectively contained within the waste containment portion 30 of the incontinence guard, in some embodiments, the outward-facing liquid-impervious layer 34 may comprise polyethylene film, polyurethane laminate (PUL), thermoplastic polyurethane (TUL), and even bioplastic. It is even contemplated that various biodegradable materials may comprise any or all of the wearer-facing liquid-pervious layer 32, outward-facing liquid-impervious layer 34, and absorbent layer. For example, plant based polymers manufactured from renewable sources such as vegetable oil may be used in manufacturing the materials instead of petroleum.

Additionally, the waste containment portion 30 may comprise a front end 36a and a rear end 36b spaced apart by opposing and elastic first and second sides 38a, 38b. Such elastic first and second sides 38a, 38b may ensure that the waste containment portion 30 closely fits the body of the wearer to form a type of seal around the wearer's crotch and legs to further prevent waste from undesirably leaking outside of the waste containment portion 30.

As may be seen with reference to FIGS. 5 through 7, the front and rear ends 36a, 36b of the waste containment portion 30 may be interchangeable with one another in some embodiments. For example, it may be seen in viewing the figures that one embodiment of the waste containment portion is bilaterally symmetrical. As such, either of the ends 36a, 36b may be placed to correspond with the wearer's lower abdomen or lower back. Still, in other embodiments, it may be desirable to form the waist containment portion 30 so that one of the ends 36a, 36b is formed to particularly correspond and conform to the wearer's lower back and buttocks while the other is formed to particularly correspond to and fit the wearer's pubis. One skilled in the art will recognize that either embodiment, and variations of the same, are sufficient to practice the invention. Thus the foregoing is offered for the sake of enabling the disclosure only and not to limit it.

The absorbent layer housed between the wearer-facing liquid-pervious layer 32 and outward-facing liquid-impervious layer 34 may comprise naturally absorbent pulps and polymers known to those skilled in the art. For example, sodium polyacrylate is known to absorb liquid on the order of about ten times its own weight. In other embodiments, the absorbent layer may comprise any or a combination of bamboo, cotton, hemp, modal, or even polyester.

In some embodiments, each element of the girdle 10 and waste containment potion 30 may comprise a material or combination of materials configured to withstand repeated washing and use. Thus, in some embodiments, materials may be chosen not for their biodegradability and cost efficient disposability, but particularly for their ruggedness.

Of course, it will be recognized by one skilled in the art that materials discussed above are offered by way of example only and not of limitation. Various materials and combinations of materials are available to construct incontinence guards and may be chosen based on various needs of those consuming such guards.

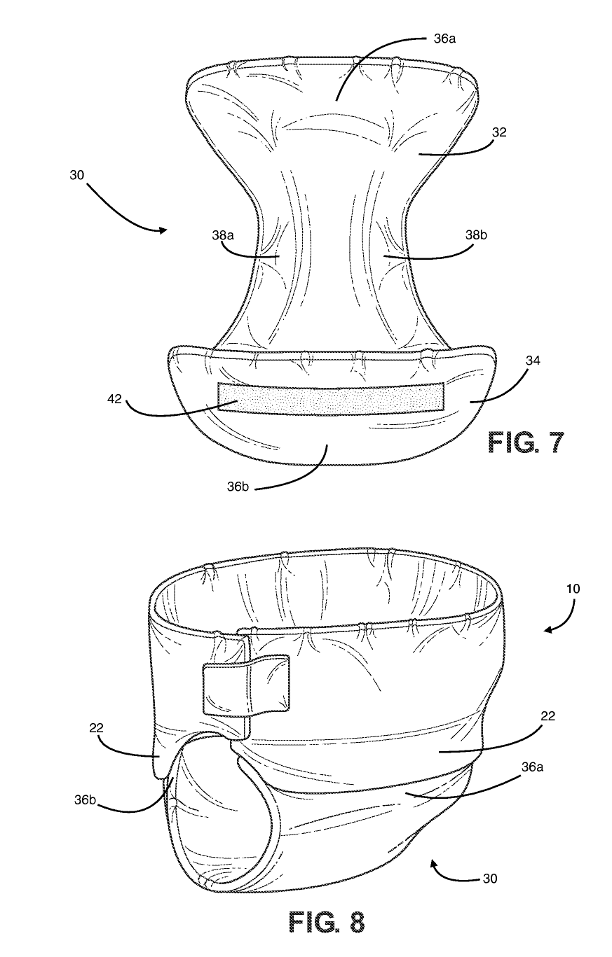

With reference now to FIGS. 8 and 9 embodiments are illustrated to show the incontinence guard in use. In some embodiments, and with particular attention to FIG. 8, the waste containment portion 30 and girdle 10 comprise wholly separate pieces that are releasably securable to one another. Briefly referring again to FIGS. 6 and 7, the waste containment portion 30 may comprise means for releasably securing 42 the outward-facing liquid-impervious side 34 of the waste containment portion 30 to the wearer-facing side of the one or more downward facing flaps formed on the girdle. Indeed, such means 42 may correspond to the means shown in FIG. 1 at character reference 24. More particularly, means for releasably securing the one or more flaps and waste containment portion 24, 42 may comprise at least one of complementary and opposing hook and loop fasteners, hook and eye closures, and snaps. In other embodiments, the means 24, 42 may comprise releasable adhesive tape. Indeed, it will be recognized by one skilled in the art that any combination of means sufficient to releasably secure the downward facing flap or flaps 22 to the waste containment portion 30, so that the waste containment portion 30 may be forcibly maintained against the body of the wearer are contemplated.

It should be noted that although the means for releasably securing 42 the outward facing liquid-impervious side 34 of the waste containment portion 30 to the wearer-facing side of the one or more downward facing flaps is shown as an elongated, rectangular strip, such means 42 may instead be formed as a swath or additional layer to cover the either of the ends 36a, 36b of the outward facing side 34 waste containment portion 30. It is contemplated that providing the means 42 in this manner may allow a parent or caregiver to more easily adjust the securement of the waste containment portion 30 to the girdle so that the incontinence guard may more closely, comfortably, and securely fit the wearer.

Thus, and with reference again to FIG. 8, the first and second ends 36a, 36b of the waste containment portion 30 may be releasably secured to the one or more downward-facing flaps 22 of the girdle 10. When the waste containment portion 30 becomes soiled with bodily waste, a wearer or caregiver of the wearer may simply detach the waste containment portion 30 from the girdle 10 and replace it with an unsoiled waste containment portion 30. This may be done regardless of the physical position taken by the wearer. Indeed, it is contemplated that arranging the elements of the incontinence guard according to the various embodiments permits a caregiver or the wearer himself to replace soiled garments with little effort. In the event that a wearer is a baby or toddler, or even pet, the caregiver may replace a soiled garment regardless of the position that the wearer has taken. For instance, the wearer may be supine, prone, standing, turned sideways with respect to the caregiver, still, moving, unable to articulate his joints, or any other position while replacing the waste containment portion 30.

Additionally, it should be noted that although two flaps 22 are shown, one, two, or even more downward-facing flaps 22 are contemplated. Moreover, one skilled in the art will recognize that the downward-facing flaps may be formed as different shapes and even placed in different positions along the bottom edge 18 of the girdle 10. For example, in some embodiments, the flaps 22 may be placed in line with either or both of a wearer's low back and lower abdomen. In other embodiments the flaps 22 may be formed to extend downwardly in line with both the front side and back side of each of the wearer's legs. In such embodiments it is contemplated that means for releasably securing 24 the girdle 10 and waste containment 30 portions may extend along a portion or the entirety of the perimeter of the waste containment portion 30. The flaps may even define a loop, or stirrup 23 as in illustrated in FIG. 4B, sized to receive one each of the wearer's legs so that the sides 38a, 38b of the waste containment portion 30 may be even more secured against the body of the wearer. The waste containment portion 30 may even be adapted to secure itself which ever form of flaps 22 or stirrups 23 might be provided. For example, in some embodiments, removable or permanent winged tabs or flaps may extend from the crotch portion of the waste containment portion 30 to securely grip the stirrups 23. Thus, the flaps 22 and even shape of the waste containment portion 30 are not limited to the placement and configuration illustrated, and such illustrations are offered by way of example only, and not of limitation.

An alternative embodiment is shown in FIG. 9. More particularly, the rear end 36b of the waste containment portion 30 may be wholly attached along the bottom edge 18 of the girdle 10. Thus, the front portion 36a of the waste containment portion 30 may be releasably securable to the girdle 10 via the one or more downward facing flaps 22 so that when soiled, the front end 36a of the waste containment portion 30 may be released from securement to the girdle 10, an absorbent and removable pad may be taken from within the waste containment portion 30 such as through pocket 46 formed in the wearer-facing liquid-pervious layer 32, and an unsoiled absorbent pad may be placed in its stead, all while avoiding a need to remove any portion of the incontinence guard from the wearer. Although the pocket 46 is shown on only a portion of the wearer-facing layer 32, it should be understood that the pocket may be disposed over substantially the entire wearer facing layer 32 of the waste containment portion 30. Indeed, it will also be understood that although shown in line with one of the sides 38a of the waste containment portion 30, the pocket 46 may also be formed in line with either of the front and rear ends 36a, 36b. It should even further be noted that the pocket 46 may be eliminated altogether. For instance, in some embodiments, the absorbent layer may be removeably adhesible to or meshable with any wearer-facing side of the waste containment portion 30 so that, when soiled, the absorbent layer (not pictured) may simply be peeled off of the waste containment portion 30. Once removed, the soiled absorbent layer may then replaced, in like fashion, with an unsoiled absorbent layer.

Thus, in any embodiment, the soiled portion of the incontinence guard only—namely the absorbent layer—may be replaced with a fresh one independent of a need to entirely remove the incontinence guard, or even the girdle 10, from the wearer's body. It is contemplated that this may permit reuse of certain portions of the incontinence guard, such as the girdle 10 and waste containment portion 30, decreasing cost to the consumer and environmental waste all while easing any difficulties associated with replacing soiled incontinence guards.

One skilled in the art will recognize that an embodiment in which the rear end 36b only, rather than the front end 36a, may be releasably securable to the one or more flaps 22 of the girdle 10 without departing from the invention. As such, the foregoing description and corresponding drawing is offered by way of example only to enable the incontinence guard, and not of limitation.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### CONCLUSIONS, RAMIFICATIONS, AND SCOPE

While certain embodiments of the invention have been illustrated and described, various modifications are contemplated and can be made without departing from the spirit and scope of the invention. For example, various aesthetic features such as color and design may be added to the layers of the waste containment portion and girdle. Accordingly, it is intended that the invention not be limited, except as by the appended claim(s).

The teachings disclosed herein may be applied to other systems, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the improved incontinence guard with which that terminology is associated. In general, the terms used in the following claims should not be constructed to limit the improved incontinence guard to the specific embodiments disclosed in the specification unless the above description section explicitly define such terms. Accordingly, the actual scope encompasses not only the disclosed embodiments, but also all equivalent ways of practicing or implementing the disclosed apparatus. The above description of embodiments of the improved incontinence guard is not intended to be exhaustive or limited to the precise form disclosed above or to a particular field of usage.

While specific embodiments of, and examples for, the method, system, and apparatus are described above for illustrative purposes, various equivalent modifications are possible for which those skilled in the relevant art will recognize.

While certain aspects of the method and system disclosed are presented below in particular claim forms, various aspects of the apparatus are contemplated in any number of claim forms. Thus, the inventor reserves the right to add additional claims after filing the application to pursue such additional claim forms for other aspects of the improved incontinence guard.

What is claimed is:

1. An incontinence guard, comprising:
a layered waste containment portion having a front end and a rear end spaced apart by opposing and elastic first and second sides, the layers comprising a wearer-facing liquid-pervious layer, an outward-facing liquid-impervious layer, and an absorbent layer between the liquid pervious and impervious layers;
a girdle sized to encircle a wearer's waist, the girdle having a wearer-facing side, an outward-facing side, a top edge, a bottom edge, and one or more flaps extending downward from the bottom edge; and
means for releasably securing the outward-facing liquid-impervious layer of at least one of the front or rear ends of the waste containment portion to the wearer-facing side of the one or more flaps.

2. The incontinence guard of claim 1, further comprising means for releasably securing any other of the front or rear end of the outward-facing liquid-impervious layer of the waste containment portion to the wearer-facing side of the one or more flaps so that the girdle and waste containment portion comprise wholly separable pieces.

3. The incontinence guard of claim 1, wherein at least one of the front or rear ends of the waste containment portion is permanently secured to the bottom edge of the girdle.

4. The incontinence guard of claim 1, wherein means for releasably securing the one or more flaps and waste containment portion comprise at least one of hook and loop fasteners and adhesive tape.

5. The incontinence guard of claim 1, wherein the absorbent layer is removable from between the liquid-pervious and impervious layers and wherein the wearer-facing liquid-pervious layer further comprises a slot sized to receive the removable absorbent layer.

6. The incontinence guard of claim 1, wherein the absorbent layer is configured to be any of removably adhesible and removably meshable to the wearer-facing liquid pervious layer of the waste containment portion.

6. The incontinence guard of claim 1, wherein the girdle comprises elastic material to forcibly maintain the front and rear ends of the waste containment portion against the body of the wearer.

7. The incontinence guard of claim 1, wherein the girdle further comprises a first end longitudinally spaced from a second end, and at least one means for adjustably securing the first end and second end to one another around the wearer's waist.

8. The incontinence guard of claim 7, wherein the at least one means for adjustably securing first and second ends of the girdle comprise any of meshable, mateable, or adhesive tabs.

9. The incontinence guard of claim 8, wherein the tabs are integrated into the body of the girdle.

10. The incontinence guard of claim 8, wherein the tabs elastically extend from the first and second ends of the girdle.

11. The incontinence guard of claim 1, wherein a single flap is fully integrated into the body of the girdle so that the girdle and flap form a continuous unit.

12. The incontinence guard of claim 1, wherein the one or more flaps elastically extend downward from the bottom edge of the girdle.

13. The incontinence guard of claim 1, wherein the waste containment portion and girdle comprise rugged material configured to withstand repeated machine washing.

14. An incontinence guard, comprising:
a girdle sized to encircle a wearer's waist, the girdle having a wearer-facing side, an outward-facing side, a top edge, a bottom edge, and one or more flaps extending downward from the bottom edge, the girdle further comprising elastic material that forcibly maintains the front and rear ends of the waste containment portion against the body of the wearer; and
a layered waste containment portion having wearer-facing liquid-pervious layer, an outward-facing liquid-impervious layer, and absorbent layer, a front end, and a rear end, the front and rear ends spaced apart by opposing and elastic first and second sides, one of the front or rear end permanently secured to the bottom edge of the girdle; and
means for releasably securing the other of the front or rear end of the outward-facing liquid-impervious layer of the waste containment portion to the wearer-facing side of the one or more flaps.

15. The incontinence guard of claim 14, wherein the one or more flaps elastically extend downward from the bottom edge of the girdle.

16. The incontinence guard of claim 14, wherein a single flap is fully integrated into the body of the girdle so that the girdle and flap form a continuous unit.

17. The incontinence guard of claim 1, wherein means for releasably securing the one or more flaps and waste containment portion comprise at least one of hook and loop fasteners and adhesive tape.

18. The incontinence guard of claim 14, wherein the waste containment portion and girdle comprise rugged material configured to withstand repeated machine washing.

19. The incontinence guard of claim 14, wherein the absorbent layer is between the liquid-pervious and impervious layers, and wherein the wearer-facing liquid-pervious layer further comprises a slot sized to receive the removable absorbent layer.

20. The incontinence guard of claim 14, wherein the absorbent layer is any of removably adhesible and removably meshable with the wearer-facing liquid-pervious layer of the waste containment portion.
